(19) United States
(12) Patent Application Publication
(10) Pub. No.: US 2018/0353090 A1
(43) Pub. Date: Dec. 13, 2018

(54) Adaptive Heart Rate Estimation

(71) Applicant: Huami Inc., Mountain View, CA (US)

(72) Inventor: Joseph Munaretto, Mountain View, CA (US)

(21) Appl. No.: 15/621,652

(22) Filed: Jun. 13, 2017

(51) Int. Cl. A61B 5/021; A61B 5/0225; A61B 5/0285; A61B 5/026

(52) U.S. Cl. CPC A61B 5/02125; A61B 5/026; A61B 5/0285; A61B 5/02255

(57) ABSTRACT

Method, apparatus and system for estimating heart rate with a wearable device. The method includes receiving movement data and heart rate data, the movement data indicative of physical exertion of an individual associated with the wearable device, and the heart rate data measured for the individual during the same period; determining an estimated human power output based on the movement data indicative of physical exertion of the individual; determining a heart rate demand value for improving a heart rate estimate based on the estimated human power output and at least one adaptive parameter, wherein the heart rate estimate corresponds to the heart rate data, and the at least one adaptive parameter is adjustable based on the heart rate demand value and the heart rate estimate; and determining an improved heart rate estimate for the individual based on the heart rate demand value and the heart rate estimate.

### TECHNICAL FIELD

This disclosure relates in general to methods, apparatuses and systems of adaptive heart rate estimation using wearable devices.

### BACKGROUND

Wearable devices are becoming increasingly popular. These can include wristbands, watches, headsets, e.g., earpieces, augmented reality (AR) or virtual reality (VR) headsets. They may be used in a variety of contexts, such as to monitor a person's health by measuring vital signals, track exercise and fitness progress, check emails or social media accounts, etc. In certain applications, wearable devices may be configured to measure and monitor signals indicative of the person's heart rate, for example.

### SUMMARY

Disclosed herein are aspects of implementations of methods, apparatuses, and systems for adaptive heart rate estimation.

In an aspect, a method for estimating heart rate with a wearable device is disclosed. The method includes receiving movement data and heart rate data, the movement data indicative of physical exertion of an individual associated with the wearable device, and the heart rate data measured for the individual during the same period; determining an estimated human power output based on the movement data indicative of physical exertion of the individual; determining a heart rate demand value for improving a heart rate estimate based on the estimated human power output and at least one adaptive parameter, wherein the heart rate estimate corresponds to the heart rate data, and the at least one adaptive parameter is adjustable based on the heart rate demand value and the heart rate estimate; and determining an improved heart rate estimate for the individual based on the heart rate demand value and the heart rate estimate.

In another aspect, a wearable device is disclosed. The wearable device includes a body configured to be coupled to a portion of an individual, a non-transitory memory, and a processor configured to execute instructions stored in the non-transitory memory to receive movement data and heart rate data, the movement data indicative of physical exertion of an individual associated with the wearable device, and the heart rate data measured for the individual during the same period; determine an estimated human power output based on the movement data indicative of physical exertion of the individual; determine a heart rate demand value for improving a heart rate estimate based on the estimated human power output and at least one adaptive parameter, wherein the heart rate estimate corresponds to the heart rate data, and the at least one adaptive parameter is adjustable based on the heart rate demand value and the heart rate estimate; and determine an improved heart rate estimate for the individual based on the heart rate demand value and the heart rate estimate.

In another aspect, a system is disclosed. The system includes a measurement component and an analysis component. The measurement component includes a body configured to be coupled to a portion of an individual, a motion sensor coupled to the body configured to measure movement data, and a heart rate sensor coupled to the body configured to measure heart rate data. The analysis component includes a non-transitory memory and a processor configured to execute instructions stored in the non-transitory memory to receive the movement data and the heart rate data measured for the individual during the same period; determine an estimated human power output based on the movement data; determine a heart rate demand value for improving a heart rate estimate based on the estimated human power output and at least one adaptive parameter, wherein the heart rate estimate corresponds to the heart rate data, and the at least one adaptive parameter is adjustable based on the heart rate demand value and the heart rate estimate; and determine an improved heart rate estimate for the individual based on the heart rate demand value and the heart rate estimate.

These and other aspects of the present disclosure are disclosed in the following detailed description, the appended claims, and the accompanying figures. Details of these implementations, modifications of these implementations and additional implementations are described below.

### BRIEF DESCRIPTION OF THE DRAWINGS

The disclosed technology is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. The description herein makes reference to the accompanying drawings, wherein like reference numerals refer to like parts throughout the several views.

FIG. 1 shows an example of a wearable device worn by an individual.

FIG. 2 is a diagram of an example implementation of a wearable device usable within implementations of the disclosure.

FIG. 3 is diagram of an example implementation of a computing device usable within implementations of this disclosure.

FIG. 4 is a diagram showing an implementation of a process for adaptive heart rate estimation using a wearable device.

FIG. 5 is a diagram showing another implementation of a process for adaptive heart rate estimation using a wearable device.

FIG. 6A is an example diagram showing levels of estimated human power output during an activity based on data collected by a wearable device.

FIG. 6B is an example diagram showing levels of estimated human power output during another activity based on data collected by a wearable device.

FIG. 7 is an example diagram showing heart rate estimates for the activity of cycling.

FIG. 8 is an example diagram showing improved heart rate estimates for the activity of cycling according to implementations of this disclosure.

FIG. 9 is an example diagram showing heart rates as a function of velocity and gradient.

### DETAILED DESCRIPTION

Example implementations of the present disclosure will be described below with reference to the accompanying drawings. The same numbers across the drawings set forth in the following description represent the same or similar elements, unless differently expressed. The implementations set forth in the following description do not represent all implementations or embodiments consistent with the present disclosure; on the contrary, they are only examples of apparatuses and methods in accordance with some aspects of this disclosure as detailed in the claims.

As mobile health care market size keeping growing, devices and systems using wearable technologies to aid fitness or health assessments become more widely used. Wearable devices, such as smart watches and fitness bands, have been used for monitoring health status and tracking fitness of individuals. Wearable devices can be used for a variety of applications, such as step counting, activity tracking or calorie-burn estimation. Activity tracking can include, for example, sleep or exercise tracking.

Among those technologies, mobile or wearable cardiac care devices and systems gain various applications, such as heart disease monitoring and engagement, biometric identification, and fitness tracking. The wearable devices can use various signals measured by various heart rate sensors as input, such as, for example, an electrocardiogram (ECG) signal and/or a photoplethysmogram (PPG) signal. Other sensors can also be used during the operation for additional input, such as a motion sensor.

This disclosure relates to systems, apparatuses, and methods for estimating heart rate with a wearable device. Movement data indicative of physical exertion of an individual associated with the wearable device is used to determine an estimated human power output, which can be used to infer the physical exertion level of the individual. Such movement data can include one or more of velocity, speed, position, altitude, and acceleration, for example. Heart rate data measured for the individual during the same period, such as PPG signal data, is used to determine a heart rate estimate. The heart rate estimate is often inaccurate due to motion artifacts that are difficult to overcome.

According to implementations of this disclosure, based on the estimated human output, a heart rate demand value is determined using an adaptive model. For example, the heart rate demand value can indicate the heart rate suitable for sustaining the activity currently performed by the individual with aerobic respiration. The heart rate demand value is used to improve the heart rate estimate from the heart rate data. An improved heart rate estimate can be determined for the individual based on the heart rate demand value and the heart rate estimate. The improved heart rate estimate can be determined using, for example, various sensor fusion techniques. The adaptive model includes at least one adaptive parameter, which is adjustable based on the heart rate demand value and the heart rate estimate (or improved heart rate estimate), which can be provided as feedback to the adaptive model over time. Other details are described herein after first describing an environment in which the disclosure may be implemented.

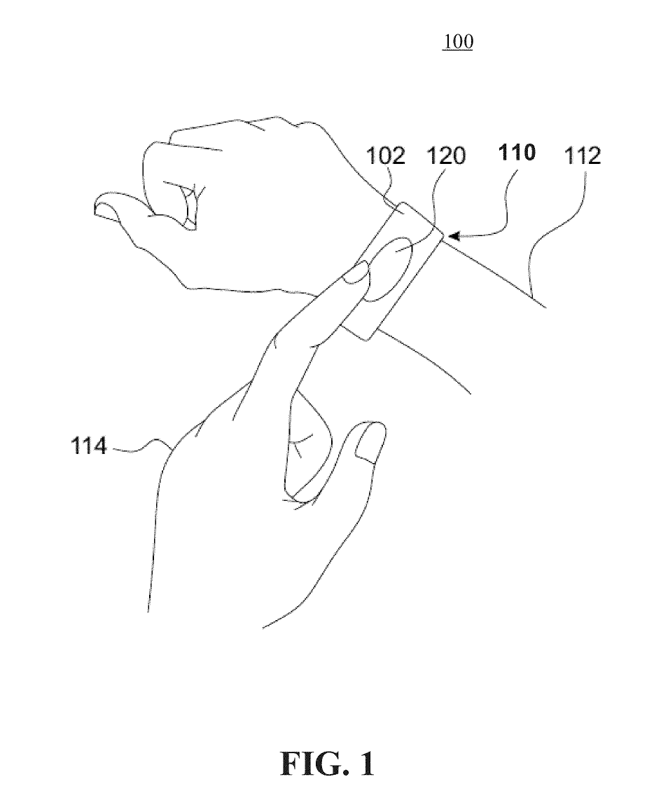

FIG. 1 is a diagram of an implementation of a wearable system 100 which can include a wearable device 110 in use. In this example, the wearable device 110 is worn by an individual on the wrist. The wearable device 110 can include a housing in the form of a ring, bracelet, wristband, watch, pendant, armband, anklet, headband, headset, belt, necklace, glove, a chest patch, or other mechanism for securing or attaching the wearable device 110 to the individual. In this example, the housing is a band 102.

In some implementations, the wearable device 110 can include one or more processing cores (such as device core 120) that are configured to receive signals from sensors (not shown) that can measure motion and/or heart rate. The signals received by the wearable device 110 can include movement data, which can include measurement data from one or more motion sensors indicating a motion status of the individual. The one or more motion sensors can be one or more devices or modules that can measure spatial and/or temporal parameters of the motion (e.g., velocity, speed, or acceleration). For example, the motion sensor can be an accelerometer, a gyroscope, an inertia measurement unit (IMU) sensor, a magnetometer, a barometric pressure sensor, an electromyography (EMG) sensor, a GPS (Global Positioning System) sensor. In one example, the motion sensor can be a 3-axis accelerometer, and the motion data received from the motion sensor can be, for example, three-dimensional accelerometer data.

Further, the wearable device 110 can exchange (such as send and/or receive) data from a remote data source. For example, a physiological profile of a user, which can be used to identify the individual, can be sent to a remote cloud server where the measurements can be stored for later retrieval and use.

Though illustrated as a single device, the wearable device 110 can be part of a wearable system 100 which can include multiple devices, or with a remote computing device such as a server that can store the signals, including the sensor data. The wearable device 110 can be a single piece wearable device, or it can include several detachable components. For example, the wearable device 110 can include a chest patch or a wristband, which can be attached to the chest or wrist of the individual. A device core which can be attached and removed from the chest patch or wristband. The chest patch can be, for example, an adhesive patch, a sticker, or the like. Further, when the wearable device 110 is activated, the wearable device 110 can, for example, monitor activities (such as eating or sleep), count steps, and/or determine heart related measurements such as heart rates or heart rate variability (HRV).

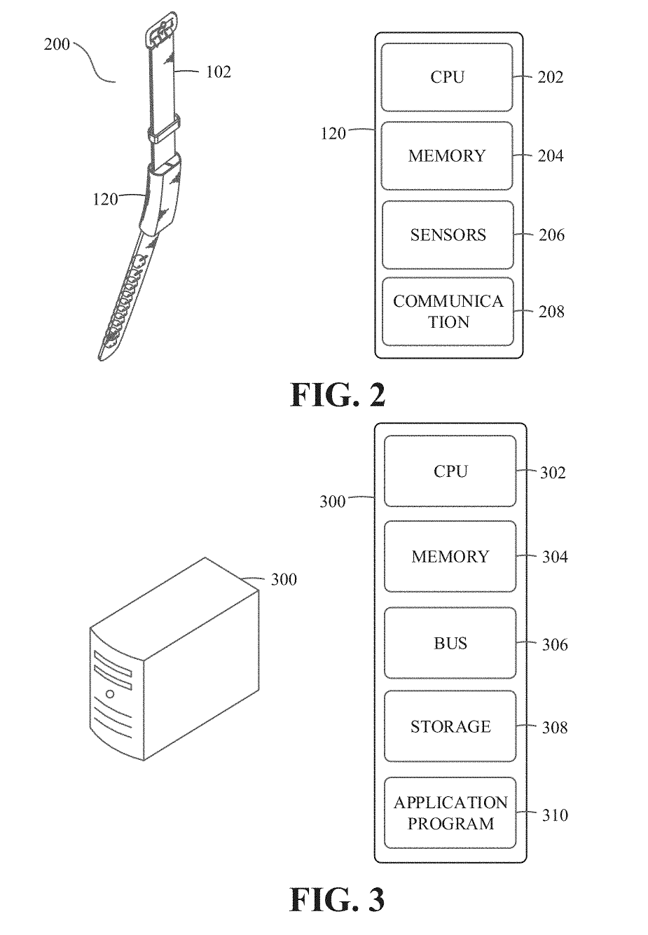

FIG. 2 is a diagram of an implementation of a wearable device 200 usable within implementations of the disclosure. The wearable device 200 can be used in the wearable system 100 discussed above with respect to FIG. 1. For example, the wearable device 200 can include a device core 120 and one or more accessory components as housing, such as a band 102 or a chest patch. The device core 120 can be integrated with the band 102, such as in the example of the wearable device 200 of FIG. 2. The device core 120 can also be removably attached to the band 102, such as in the example of the wearable device 110 of FIG. 1.

In an implementation, the device core 120 comprises one or more of CPU 202, memory 204, sensor 206, communication 208, or other components. One example of CPU 202 is a central processing unit. CPU 202 may include single or multiple processors each having single or multiple processing cores. Although implementations of wearable device 200 can be practiced with a single CPU as shown, advantages in speed and efficiency may be achieved using more than one CPU.

Memory 204 can comprise random access memory (RAM), flash memory, read-only memory (ROM), or any other suitable type of storage device. Memory 204 may include executable instructions and data for access by CPU 202, such as data generated from sensors 206. Alternatively, memory 204 may include another type of device, or multiple devices, capable of storing data for processing by CPU 202. CPU 202 may access and manipulate data in memory 204 via a bus (not shown).

Sensors 206 can be one or more sensors disposed within or otherwise coupled to wearable device 200, for example, for identifying, detecting, determining, or otherwise generating signal data indicative of measurements associated with wearable device 200. For example, sensors 206 can comprise one or more electromyography sensors, accelerometers, barometric pressure sensors, receivers with antennas, cameras, light emitters, touch sensors, heart rate sensors, or the like. Receivers with antennas can include, for example, GPS (Global Positioning System) sensors. The cameras can be RGB cameras, infrared cameras, monochromatic infrared cameras, or any other suitable cameras. The light emitters can be infrared light emitting diodes (LED), infrared lasers, or any other suitable lights.

When the wearable device is worn by an individual, one or more of sensors 206 can be used to measure motion and/or physiological states of the individual. For example, sensors 206 can include one or more motion sensors, which can be an accelerometer, a gyroscope, a magnetometer, an inertia measurement unit (IMU) sensor, a barometric pressure sensor, a GPS sensor, or a combination of the above. The motion sensor(s) can have one or more measurement axes (e.g., 3, 6, 9 or any other number), for measuring dimensions or directions. For example, the motion sensor from which the motion is collected can be a motion sensor capable of measuring acceleration in three spatial dimensions (e.g., x-, y-, and z-direction), such as a 3-axis accelerometer.

Motion sensor(s) can measure spatial and/or temporal parameters of motion. Data collected by the motion sensor(s) can be used to determine movement data, which can include, for example, one or more of velocity, speed, position, altitude, and acceleration. Movement data can be determined using a single sensor or multiple sensors. For example, a GPS sensor can be used to determine velocity (e.g., running speed in a trail run). In another example, the GPS sensor and a barometric pressure sensor can be used together to determine the change in altitude. Movement data can be used to infer the physical exertion level of the individual, as will be discussed in further detail below.

Sensors 206 can comprise one or sensors for measuring physiological states of the individual such as heart rate (“heart rate sensors”). Examples of heart rate sensors include, for example, an electrocardiography (ECG) sensor, a photoplethysmogram (PPG) sensor, a pulse oximeter, an infrared (IR) sensor, or the like.

Sensors 206 can also comprise one or more bioimpedance sensors, microphones, temperature sensors, touch screens, finger readers, iris scanners, a combination of the above, or the like. Implementations of sensors 206 can include a single sensor, or any suitable sensor combinations. Signal data can be identified, detected, determined, or otherwise generated based on any single sensor or combination of sensors included in wearable device 200. In some implementations, some of the signal data can also be generated by another device such as computing device 300.

Communication 208 can be a hardware or software component configured to communicate data (e.g., measurements, etc.) from sensors 206 to one or more external devices, such as another wearable device or a computing device, for example. In an implementation, communication 208 comprises an active communication interface, for example, a modem, transceiver, transmitter-receiver, or the like. In an implementation, communication 208 comprises a passive communication interface, for example, a quick response (QR) code, Bluetooth identifier, radio-frequency identification (RFID) tag, a near-field communication (NFC) tag, or the like. Communication 208 can operate over wired or wireless communication connections, such as, for example, a wireless network connection, a Bluetooth connection, an infrared connection, a NFC connection, a cellular network connection, a radio frequency connection, or any combination thereof. In some implementations, communication 208 can use sound signals as input and output, such as, for example, an ultrasonic signal or a sound signal via an audio jack. Implementations of communication 208 can include a single component, one of each of the foregoing types of components, or any combination of the foregoing components.

In some implementations, some or all the components described above can be included as part of the housing, such as the band 102 in FIG. 2. For example, a sensor or a communication component can be included as part of the band 102.

Wearable device 200 can also include other components not shown in FIG. 2. For example, wearable device 200 can include one or more input/output devices, such as a display. In an implementation, the display can be coupled to CPU 202. In an implementation, other output devices may be included in addition to or as an alternative to the display. When the output device is, or includes a display, the display may be implemented in various ways, including by a LCD, CRT, LED, OLED, etc. In an implementation, the display can be a touch screen display configured to receive touch-based input, for example, in manipulating data output to the display.

FIG. 3 shows an example computing device 300 usable within implementations of this disclosure. An example block diagram of a configuration of computing device 300 is also shown. Computing device 300 can be a part of the wearable system 100 for adaptive heart rate estimation. In some implementations, computing device 300, wearable device 110 or 200, or any device having measurement capabilities, can be the same device. Computing device 300 can be implemented by any configuration of one or more computers, such as a microcomputer, a mainframe computer, a super computer, a general-purpose computer, a special-purpose/dedicated computer, an integrated computer, a database computer, a remote server computer, a personal computer, a laptop computer, a tablet computer, a cell phone, a personal data assistant (PDA), a wearable computing device, e.g., a smart watch, or a computing service provider, e.g., a website host, or a cloud service provider. In some implementations, computing device 300 can be a smart phone device that can be used to display and analyze signals such as movement and heart rate data. In some implementations, certain operations described herein can be performed by a computer (e.g., a server computer) in the form of multiple groups of computers that are at different geographic locations, which may communicate with one another by way of, such as, a network. While certain operations can be shared by multiple computers, in some implementations, different computers can also be assigned with different operations.

Computing device 300 can include at least one processor such as CPU 302. CPU 302 as well as CPU 202 can be any type of device, or multiple devices, capable of manipulating or processing information such as signals or other data. CPU 302 can be distributed across multiple computing devices.

Memory 304 as well as memory 204 can be, for example, a random access memory device (RAM), a read-only memory device (ROM), an optical disc, a magnetic disc, or any other suitable type of storage device, and can store code and data that can be accessed by CPU 302 using a bus 306. Although a single bus 306 is depicted, multiple buses can be utilized. Memory 304 can be distributed across multiple machines or devices such as network-based memory or memory in multiple machines performing operations that can be described herein as being performed using a single computing device for ease of explanation. The code can include an operating system and one or more application program 310 processing and/or outputting the data. As will be discussed in detail below, application program 310 can include software components in the form of computer executable program instructions that cause CPU 302 to perform some or all of the operations and methods described herein. In some implementations, hardware configuration is used to implement computing device 300 or at least an analysis component of computing device 300, in which application program 310 stored by memory 304 can implement some or all of the processes as described in more detail below.

Computing device 300 can optionally include a storage device 308 in the form of any suitable non-transitory computer readable medium, such as a hard disc drive, a memory device, a flash drive or an optical drive. Storage device 308, when present, can provide additional memory when high processing requirements exist. Storage device 308 can also store any form of data, relating or not relating to cardiac information. Further, storage device can be a component of computing device 300 or can be a shared device that is accessed via a network.

Computing device 300 can include one or more sensors, such as the sensors described above in connection with FIG. 2. For example, computing device 300 can include a GPS sensor that can be used to determine the velocity of movement. In another example, computing device 300 can include a GPS sensor and a barometric pressure sensor that can be used to determine the change of altitude of the computing device 300.

Computing device 300 can include more devices or components. For example, computing device can further include one or more input devices, output devices, communication devices, or any other device that can be used to transmit, store, process, and present data.

Although FIG. 3 depicts one hardware configuration that can implement computing device 300, other configurations can be utilized. The hardware configuration of a computing system as depicted in an example in FIG. 3 thus can be implemented in a wide variety of configurations.

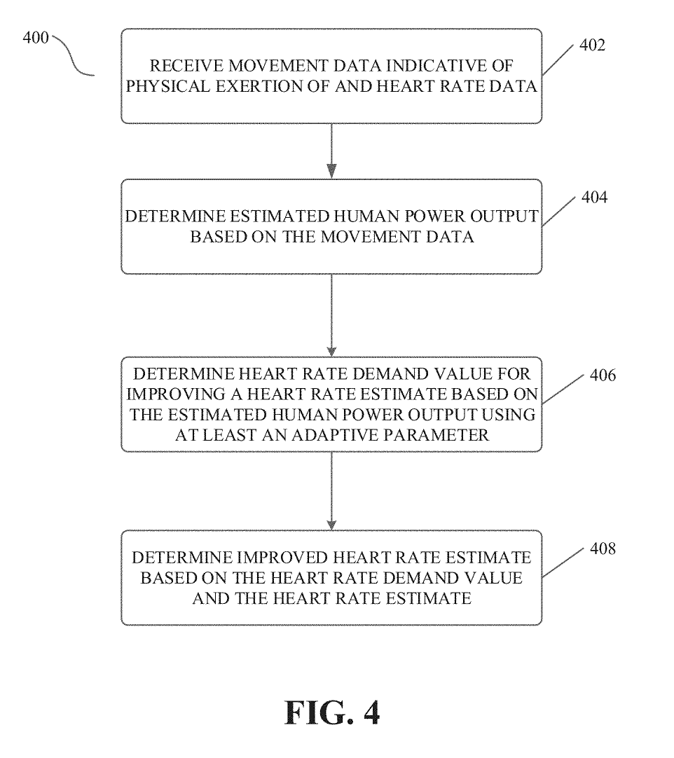

FIG. 4 is a diagram showing an example process 400 of adaptive heart rate estimation using a wearable device. In some implementations, some or all of the process 400 can be implemented in a device or apparatus such as the wearable device 110 or 200, or the computing device 300. Implementations of process 400 can be performed entirely on the wearable device (e.g., wearable device 110 or 200) on which the sensor data is collected or generated, or be performed on a computing device (e.g., computing device 300) in communication with the wearable device, or another wearable device. For example, the sensor data processing aspects of process 400 can be performed by instructions executable on computing device 300. In some implementations, some portions of process 400 can be performed by instructions executable on the computing device, and other portions of process 400 can be performed by instructions executable on one or more other devices, such as wearable device 110 or 200. In some implementations, the computing device can be a smart phone, which can receive and display signals. The computing device can also be a wearable device, such as a smart watch. In some implementations, the computing device can be a cloud server. In some implementations, the wearable device and the computing device can be the same device.

As described above, the wearable device, such as a fitness band or a smart watch, can include or be coupled to a motion sensor that can generate movement data. The motion sensor can be one or more devices or modules that can measure spatial and/or temporal parameters of the motion (e.g., velocity, speed, position, or acceleration). For example, the motion sensor can be an accelerometer, a gyroscope, a magnetometer, an inertia measurement unit (IMU) sensor, a barometric pressure sensor, a Global Positioning System (GPS) sensor, or a combination of the above. The motion sensor can have one or more measurement axes (e.g., 1, 2, 3, 6, 9 or any other number), for measuring dimensions or directions. For example, the motion sensor from which the motion is collected can be a motion sensor capable of measuring acceleration in three spatial dimensions (e.g., x-, y-, and z-direction), such as a 3-axis accelerometer.

The wearable device 200 can also include or be coupled to a heart rate sensor that can generate heart rate data. The heart rate sensor can be, for example, an electrocardiography (ECG) sensor, a photoplethysmogram (PPG) sensor, a pulse oximeter, or an infrared (IR) sensor. In some examples described below, the heart rate sensor can be a PPG sensor.

At operation 402, movement data indicative of physical exertion of an individual associated with the wearable device, and heart rate data measured for the individual during the same period are received.

In various examples, data such as the movement data and the heart rate data can be generated by a wearable device (e.g., the wearable device 110 or 200). The data can also be generated by another device and received by the wearable device. The data can also be generated by the wearable device or another device, and received by a computing device (e.g., the computing device 300).

The “receiving” used herein can refer to receiving, inputting, acquiring, retrieving, obtaining, reading, accessing, determining, or in any manner inputting data. As used herein, information, signals, or data are received by transmission or accessing the information, signals, or data in any form, such as receiving by transmission over a network, receiving by accessing from a storage device, or receiving by individual operation of an input device.

Data such as the movement data and the heart rate data can be received, for example, in data segments. Data segments can be received, for example, continuously or episodically. Data segments can be received as, for example, a continuous stream of accelerometer data. A data segment can be of any size. For example, a data segment can include data collected during twenty seconds or one minute. A data segment can bear a time stamp indicating the time period associated with the data segment.

The movement data can include measurement data from the motion sensor indicating physical exertion of the individual (e.g., acceleration, velocity, direction, position, or altitude). The movement data can be generated by a motion sensor in the wearable device, which can be used to determine motion characteristics of the wearable device, such as linear or angular motion characteristic.

The heart rate data can include measurement data from the heart rate sensor for the individual. The measurement data can be used for estimating the heart rate. For example, the heart rate sensor can be a PPG sensor. The estimated heart rate (also referred to as “heart rate estimate”) can be calculated based on the measurement data from the PPG sensor. For example, various algorithms and techniques can be used to calculate the heart rate estimate from the PPG sensor data. The heart rate estimate can occur during the same period as the movement data indicating the physical exertion.

At operation 404, an estimated human power output is determined based on the movement data indicative of physical exertion of the individual. Operation 404 can be performed by the wearable device, or on an accompanying application running on the computing device (such as a smart phone).

Estimated human power output (“power output”) can be determined based on data such as, but not limited to, body mass, velocity, position, gradients of terrain, etc., some or all of which can be associated with or derived from the movement data. In some implementations, an activity currently performed by the individual can be determined based on the movement data. Based on the activity, a physical activity model can be selected for determining the estimated human power output. The physical activity model can include at least one adaptive parameter for determining the estimate human power output. The physical activity model can be part of a power model, which includes the physical activity model and a heart rate demand model. The power model can include multiple physical activity models. The at least one adaptive parameter can be learned and adjusted as data are collected, for the individual and/or for multiple users.

For example, the following physical activity model can be used for activities such as running and cycling: P=f(m, v, i), where m represents the mass value associated with the individual, v represents the velocity of the individual, and i represents the gradient of an inclination of the terrain.

In one example, a physical activity model for running can be represented as:

Pᵣ=m*v*[c₁i+c₂i²+c₃i³+ . . . cₙiⁿ] (1)

where c₁, c₂, . . . , cₙ are coefficients that can be used to describe the polynomial relationship between gradient and power output.

In another example, a physical activity model for cycling can be represented as:

P_{c}=v*[mg*(sin(arctan(i))+Cᵣ*cos(arctan(i)))+0.5*C_{d}*A*ρ*v²] (2)

where v*mg*(sin(arctan(i))) accounts for work against gravity, v*mg*Cᵣ*cos(arctan(i)) accounts for work against rolling resistance, and v*0.5*C_{d}*A*ρ*v² accounts for work against air drag.

Initially, the physical activity models can be determined based on known models. As more data are collected, the physical activity model can be adjusted. The adjustments can be determined separately for individual users, or globally for multiple users.

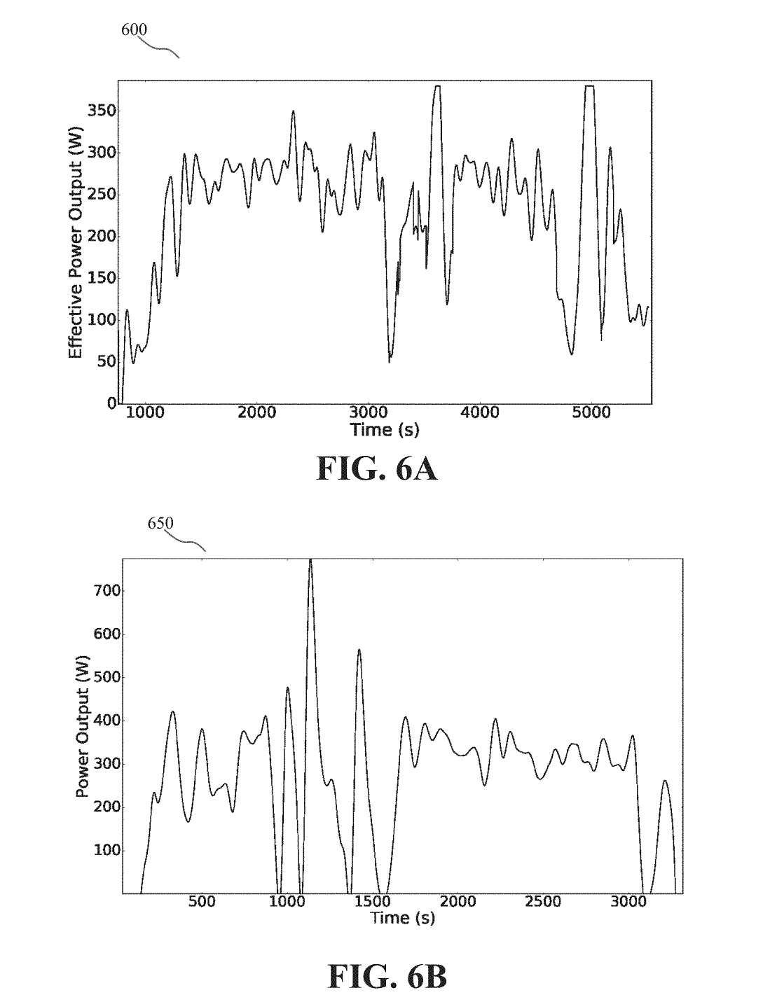

Examples of estimated power outputs for some example activities are shown in FIGS. 6A and 6B. FIG. 6A is an example diagram 600 showing levels of estimated human power output during a first activity (e.g., running) based on movement data collected by a wearable device. FIG. 6B is an example diagram 650 showing levels of estimated human power output during a second activity (e.g., cycling) based on movement data collected by a wearable device. As can be seen from the FIGS. 6A and 6B, estimated human power outputs vary for different activities, in values and over time. Therefore, in some implementations, different physical activity models are used for different activities. In some implementations, it is possible to use the same physical activity model for multiple activities. In some examples, a similar physical activity model can be used for multiple activities while setting different coefficient values for each activity. For example, the same physical activity model can be used for walking and running.

At operation 406, a heart rate demand value is determined for improving a heart rate estimate based on the estimated human power output and at least one adaptive parameter. The heart rate estimate corresponds to the heart rate data received at operation 402. For example, the heart rate estimate can be a heart rate estimate based on the PPG sensor data. The at least one adaptive parameter is adjustable based on the heart rate demand value and the heart rate estimate. The at least one adaptive parameter can be part of a heart rate demand model that is used to determine the relationship between the power output and the heart rate demand. As discussed above, the heart rate demand model can be part of the power model. The at least one adaptive parameter, or any other adaptive parameters for the power model, can be adjusted over time by personalized learning from individual users and/or global learning from multiple users.

Initially, the heart rate demand value can be determined from the estimated human power output based on known factors such as estimates of gross mechanical efficiency of the activity, average heart rate physiological ranges, and user fitness. For example, if it is known that cycling efficiency is around 20-22%, the human power output for cycling can be converted into a user energy expenditure. Based on known heart rate ranges and user fitness, a general estimate of heart rate can be determined.

In some implementations, determining the heart rate demand value can include determining the heart rate demand value based on the estimated human power output, a maximum heart rate, a rest heart rate, and a fitness level of the individual. The at least one adaptive parameter can include a scaling parameter related to human power output produced at maximum oxygen effect. The fitness level can be determined based on the estimated human power output and the scaling parameter. The scaling parameter can be adjusted for the individual over time.

For example, Heart Rate Reserve (HRR) can be determined as the difference between rest and maximum heart rates:

HR_{R}=(HR_{Max}−HR_{Rest}) (3)

Heart rate demand value can be determined from HRR as follows:

$${HR}_{D} = \frac{{P}_{R}}{{P}_{VO 2 max}} * {HR}_{R} + {HR}_{Rest} \qquad ( 4 )$$

where P_{R} is the estimated human power output, and P_{VO2max} is a scaling parameter related to human power output produced at maximum oxygen effect.

As discussed above, initially, the power models, such as the physical activity models and the heart rate demand models, can be determined based on known models. As more data are collected, the models can be adjusted. For example, individual models of heart rate kinetics can be generated from user data over time. The at least one adaptive parameter can be adjusted based on comparing the heart rate estimate and the heart rate estimate using adaptive learning.

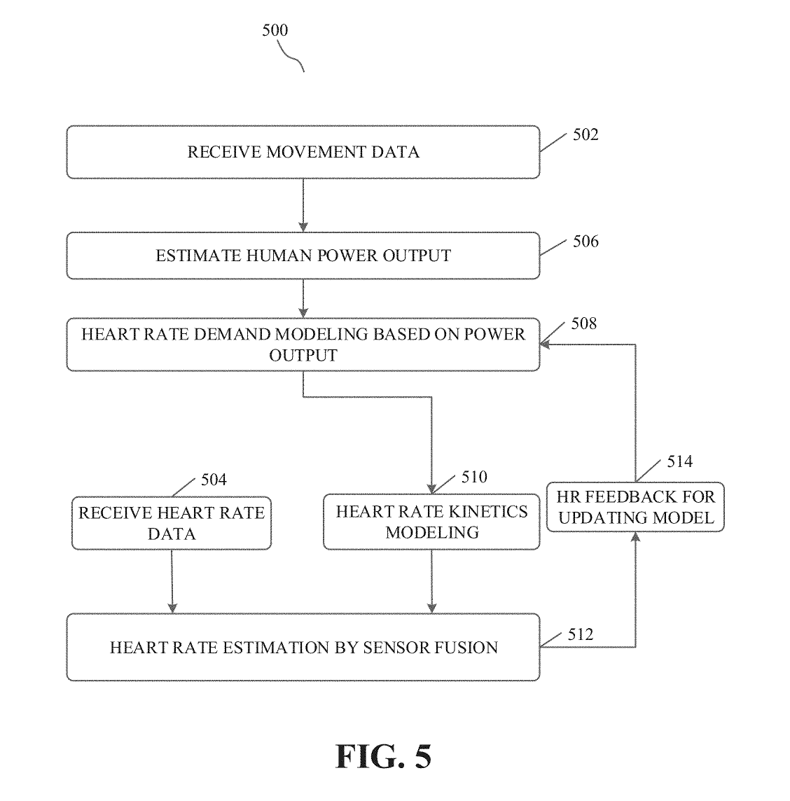

Parameters, such as maximum and rest heart rates and the scaling parameter(s) (such as PVO2max), can be determined for individual users and/or activity types, which can be stored and updated in the physiological profile(s) of the individual. These parameters can be adaptively learned and evolve over time. Techniques for determining the heart rate demand value can be represented in various forms, such as linear, non-linear, piecewise-linear equations, and so on. For example, scaling parameters such as PVO2max, α and β (to be discussed in FIG. 5), which relate to cardiac output and cardiovascular fitness (e.g., heart rate kinetics) of the individual, can be used to describe the physiological profile(s) of the individual.

Adaptive learning that personalizes the parameters can be performed over time. For instance, the polynomial coefficients in the running model can be tuned for each specific user. Failure to personalize these models can cause the heart rate estimate to be less robust (e.g. a nominal change in speed may result in a change in the heart rate estimate that is not desirable). In addition, data for a variety of activities can be aggregated for multiple users to help establish the feasible ranges of parameters, which are then used to learn the parameters for each individual user.

In some implementations, gradient descent technique can be used to optimize the values of these parameters so that the heart rate estimate derived from the physical exertion matches the heart rate estimate from the heart rate sensor, such as the PPG sensor. This can be performed with a slow learning rate, and when the PPG signal quality is high. This technique can reduce bias and variance in estimation accuracy of the power models and make them useful for sensor fusion later.

For example, application-level model enhancement can be carried out based on adaptive learning. Data segments that meet certain criteria (e.g., signal quality thresholds) can be aggregated. The criteria can also include sensor-specific criteria, such as velocity requirement for movement data (which can include both horizontal and vertical velocities). Regression, such as non-linear least square regression, can be performed on the aggregated data segments. The adaptive parameters can be updated and pushed to the device firmware.

At operation 408, an improved heart rate estimate is determined for the individual based on the heart rate demand value and the heart rate estimate. The improved heart rate estimate can be determined by (e.g., fusing) the heart rate estimate corresponding to the heart rate data (e.g., PPG sensor data) received at operation 402 and a heart rate estimate based on the heart rate demand value determined at operation 406.

For example, data from multiple sensors can be fused by filters such as Bayesian filters, Kalman filters, or Particle filters. In one example, probability distributions can be generated for each sensor. For example, probability distributions can be generated for the heart rate sensor and the motion sensor(s), such as the GPS sensor. Quality of the probability distributions can be evaluated to determine the gain factor (e.g., weights) attributed to each sensor and/or the model for the final estimation. For example, confidence metrics can be generated to evaluate the signal quality of each sensor. The quality evaluation can use, for example, variance for a normal distribution.

By integrating the heart rate estimate based on the heart rate data (e.g., PPG sensor data) with the heart rate estimate based on the power model, better heart rate estimations can be achieved by inferring additional information about potential heart rate levels from additional sensors. In addition, models used for the power output conversion, heart rate demand estimation and heart rate kinetics can be adapted and improved over time, which can occur globally for multiple users, on an individual basis, or both.

FIG. 5 is a diagram showing an example of a process 500 of estimating heart rate using heart rate kinetics modeling. In some implementations, some or all of the process 500 can be implemented as part of process 400. In some implementations, some or all of the process 500 can be implemented in a device or apparatus such as the wearable device 110 or 200, and/or the computing device 300.

Heart rate kinetics models can be implemented to predict the change in heart rate for the individual, which can be used to improve the heart rate estimate. In some implementations, such as in continuously heart rate monitoring, the improved heart rate estimate can be determined based on a predicted heart rate change and an improved heart rate estimate determined for a previous period. The predicted heart rate change can be determined based on the heart rate demand value and the at least one adaptive parameter. For example, the at least one adaptive parameter can include the scaling parameter, such as P_{VO2max}, and at least another parameter associated with a physiological profile learned from the individual, such as parameters representing personalized heart rate kinetics for the individual. The at least one adaptive parameter can include one or more adaptive parameters described above in the description of FIG. 4.

At operation 502, movement data is received from a first sensor. Similar to operation 402, the movement data received is associated with an individual wearing the wearable device, such as the wearable device 110 or 200. For example, the movement data can be collected by the first sensor, or received by the first sensor from another device. Similar to operation 402, the movement data can include, for example, accelerometer data, velocity, direction, position, altitude, or other data that is indicative of physical exertion of the individual. The first sensor can be a motion sensor. For example, the first sensor can be, for example, an accelerometer, a gyroscope, a magnetometer, an inertia measurement unit (IMU) sensor, a barometric pressure sensor, a Global Positioning System (GPS) sensor, another physical activity sensor, or a combination of the above. The movement data can include, for example, raw data received from the wearable device, or processed data such as aggregated or annotated data. Similar to operation 402, the movement data can be processed in data segments.

In some implementations, more than one sensor can be used to generate the movement data for heart rate estimation. For example, a GPS sensor and a barometric pressure sensor can be used to determine speed and change of altitude of the individual.

In some implementations, confidence metrics can be generated for the first sensor, which can be a function of the signal quality of the first sensor. The first sensor can include multiple sensors.

At operation 504, heart rate data is received from a second sensor. Similar to operation 402, heart rate data can be data collected or otherwise received by the second sensor such as, for example, an electrocardiography (ECG) sensor, a photoplethysmogram (PPG) sensor, a pulse oximeter, or an infrared (IR) sensor. Heart rate estimates can be generated from the heart rate data using existing heart rate estimation techniques. For example, PPG heart rate estimation techniques can be used to generate heart rate estimate from PPG data. In addition, a confidence or uncertainty metric can be generated for the heart rate data (e.g., PPG data), which can be a function of the signal quality of the second sensor (e.g. the PPG sensor).

At operation 506, human power output is estimated based on the movement data. Similar to operation 404, the estimated human power output can be determined based on data such as, but not limited to, body mass, velocity, position, gradients of terrain, etc., some or all of which can be associated with or derived from the movement data. In some implementations, the human power output can be determined based on the activity performed by the individual (e.g., running or cycling).

At operation 508, heart rate demand value is determined based on the human power output estimated at operation 504 (“heart rate demand modeling”). Similar to operation 406, the heart rate demand value can be determined using the adaptive parameter(s). The adaptive parameter(s) can be adjusted based on the improved heart rate estimate at operation 512 (or the heart rate estimate at operation 504, or both), which can be provided to the heart rate demand model as feedback.

At operation 510, heart rate kinetics is determined based on the heart rate demand value (“heart rate kinetics modeling”). In some implementations, such as in continuously heart rate monitoring, the improved heart rate estimate can be determined based on a predicted heart rate change and an improved heart rate estimate determined for a previous period. The predicted heart rate change is determined based on the heart rate demand value and the at least one adaptive parameter. For example, the at least one adaptive parameter can include the scaling parameter, such as P_{VO2max}, and at least another parameter associated with a physiological profile learned from the individual, such as parameters representing personalized heart rate kinetics for the individual.

For example, the predicted change in heart rate can be modeled using a differential equation. One example is as follows:

\dot{H}R=α*(HR_{D}−HR)^{β} (5)

where α and β are parameters representing personalized heart rate kinetics for the individual, HR is the current heart rate, and \dot{H}R is the estimated change in heart rate. HR_{D} is the heart rate demand value. More complex models can also be implemented to allow a to be a function of other parameters, such as resting, maximum (or current heart rate), and a model of lactate accumulation. Heart rate kinetics model can be part of the power model. As described above, the parameters PVO2max, α and β, which relate to cardiac output and cardiovascular fitness, can be included in the physiological profile(s) of the individual and adjusted over time.

The predicted change in heart rate \dot{H}R can to be used to derive the current heart rate estimate HRₜ:

HRₜ=\dot{H}R*dt+HRₜ₋₁ (6)

where HRₜ₋₁ is the HR estimate of the previous timestamp (e.g., a previous data segment).

At operation 512, heart rate estimation can be performed using the data from multiple sensors (“sensor fusion”). Similar to operation 408, the movement data and the heart rate data from the first and second sensors can be fused together to determine an improved estimated heart rate.

Confidence metrics can be generated to evaluate the signal quality of each sensor. For example, an error metric can be determined relative to the heart rate estimate from the heart rate data (e.g., PPG heart rate) when the confidence metric of the heart rate estimate is high. For example, an error metric can be determined for the heart rate sensor. Scaling parameters such as PVO2max, α and β can be updated using various techniques, such as stochastic gradient descent on Jacobian of error metric in one example. Confidence metrics can be generated for evaluating signal quality measures of the first sensor (which can include one or multiple motion sensors) and the error metric determined for the second sensor (e.g., the heart rate sensor).

Similar to operation 406, various filters can be designed and used to fuse the heart rate estimates from multiple sources. In an example using Kalman Filter, state variables for HR and \dot{H}R can be selected. Various functions can be designed, such as state transition function, process noise matrix, measurement function(s) (which can indicate the relationship between measurement and estimate), and measurement noise matrix (which can be a function of signal quality metrics of both the movement data and the heart rate data). After initial conditions are set, a Kalman Filter can be used to fuse the heart rate estimates from various sources to determine the improved heart rate estimate.

At operation 514, the improved estimated heart rate is provided as feedback to the power model (such as the heart rate demand model or the heart rate kinetics model), which can be used to adjust the parameters for the various models. For example, the parameters for the heart rate demand model (“adaptive parameter learning”) can be adjusted at operation 508.

For example, application-level model enhancement can be used to adjust the parameters. Confidence metrics can be generated for data segments, and data segments that meet certain criteria (e.g., above certain signal quality thresholds) can be identified and aggregated. Other criteria can be sensor-specific, such as requiring stationary (stable or near stationary) for heart rate data, and/or near constant for horizontal velocity from the GPS sensor and vertical velocity (e.g., incline gradient) from the GPS sensor and the barometric pressure sensor. Additional criteria can include, for example, requirements on the data segments and/or synchronization. Regression, such as non-linear least square polynomial regression, can be performed on the aggregated data segments to update the adaptive parameters, which can be pushed to the device firmware.

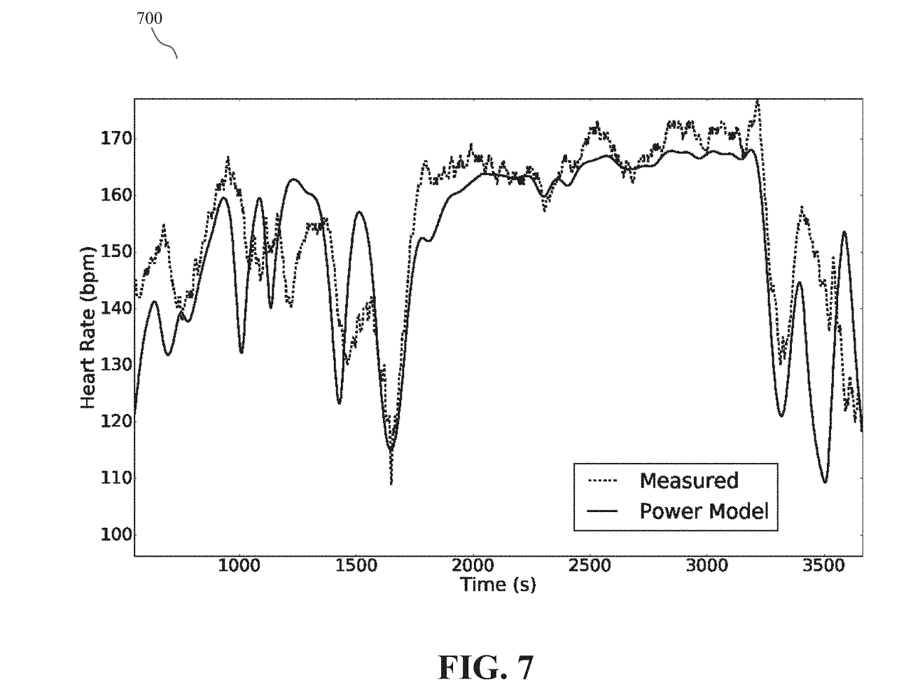

FIG. 7 is an example diagram 700 showing heart rate estimates during an activity of cycling. As shown, the dotted lines represent ground truths, such as pulse rates. The heart rate estimates determined using the power model for cycling is shown in solid lines, which roughly match the ground truths.

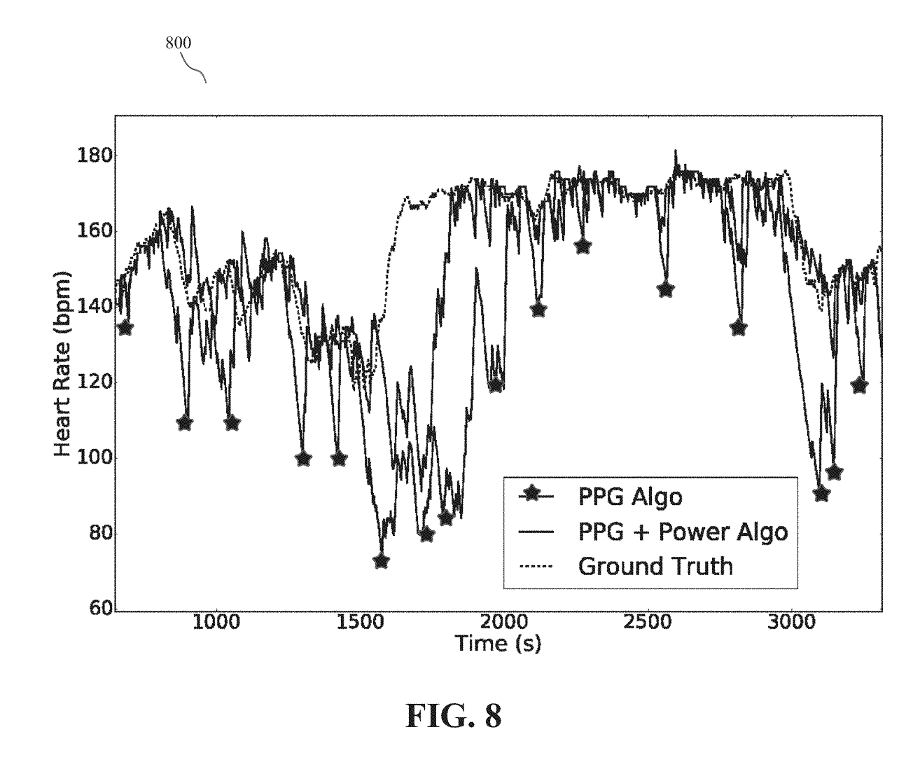

FIG. 8 is an example diagram 800 showing improved heart rate estimates for the activity of cycling and heart rate estimates based on data measured by a PPG sensor. Ground truths are represented in dotted lines. The lines with stars represent the heart rate estimates based on data measured by the PPG sensor. The solid lines (without stars) represent the improved heart rate estimates using data from both the PPG sensor and the power model. As shown, the usage of both the PPG sensor and motion sensor(s) allows for more accurate estimates of the heart rates, which are closer to the ground truths.

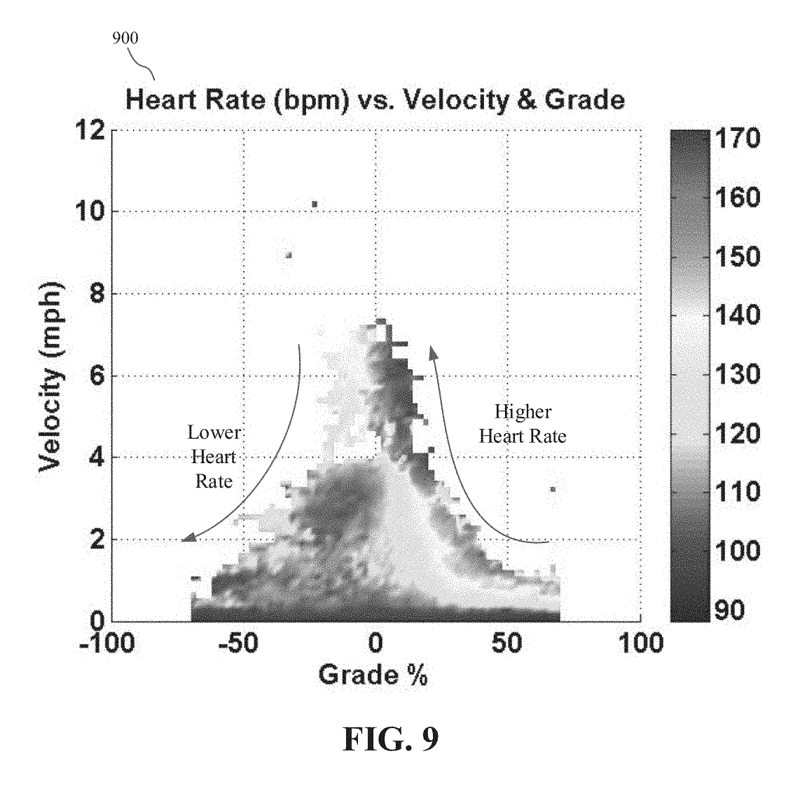

FIG. 9 is an example diagram 900 showing heart rates as a function of velocity and gradient for an individual. In this example, the individual is performing the activity of running or walking. The gradients (grades) as shown represent the gradients of the running or walking trail. When the velocity increases, the heart rate increases. When the gradient increases, the heart rate also increases. The velocity tends to peak where the gradient is around 0. At the same velocity, the heart rate tends to be higher when the gradient is higher. This shows that multiple sensors can be used to make the improved heart rate estimates more accurate. As the data is uploaded to a smartphone (or cloud), the polynomial coefficients can be updated to minimize heart rate estimation error (using techniques such as gradient descent). This data can then be uploaded to the cloud to improve the power model for multiple users.

While the current disclosure has been described in connection with certain embodiments or implementations, it is to be understood that the invention is not to be limited to the disclosed embodiments or implementations, but, on the contrary, is intended to cover various modifications and equivalent arrangements included within the scope of the appended claims, which scope is to be accorded the broadest interpretation so as to encompass all such modifications and equivalent structures as is permitted under the law.

The aspects herein can be described in terms of functional block components and various processing operations. Such functional blocks can be realized by any number of hardware and/or software components that perform the specified functions. For example, the described aspects can employ various integrated circuit components, e.g., memory elements, processing elements, logic elements, look-up tables, and the like, which can carry out a variety of functions under the control of one or more microprocessors or other control devices.

Similarly, where the elements of the described aspects are implemented using software programming or software elements the disclosure can be implemented with any programming or scripting language such as C, C++, Java, assembler, or the like, with the various algorithms being implemented with any combination of data structures, objects, processes, routines or other programming elements. Functional aspects can be implemented in algorithms that execute on one or more processors. Furthermore, the aspects of the disclosure could employ any number of techniques for electronics configuration, signal processing and/or control, data processing and the like. The words “mechanism” and “element” are used broadly and are not limited to mechanical or physical embodiments or aspects, and can include software routines in conjunction with processors and other electronic computing devices.

Implementations or portions of implementations of the above disclosure can take the form of a computer program product accessible from, for example, a computer-usable or computer-readable medium. A computer-usable or computer-readable medium can be any device that can, for example, tangibly contain, store, communicate, or transport a program or data structure for use by or in connection with any processor. The medium can be, for example, an electronic, magnetic, optical, electromagnetic, or a semiconductor device. Other suitable mediums are also available. Such computer-usable or computer-readable media can be referred to as non-transitory memory or media, and can include RAM or other volatile memory or storage devices that can change over time. A memory of an apparatus described herein, unless otherwise specified, does not have to be physically contained by the apparatus, but is one that can be accessed remotely by the apparatus, and does not have to be contiguous with other memory that might be physically contained by the apparatus.

Any of the individual or combined functions described herein as being performed as examples of the disclosure can be implemented using machine readable instructions in the form of code for operation of any or any combination of the aforementioned computational hardware. Computational code can be implemented in the form of one or more modules by which individual or combined functions can be performed as a computational tool, the input and output data of each module being passed to/from one or more further module during operation of the methods and systems described herein.

Information, data, and signals can be represented using a variety of different technologies and techniques. For example, any data, instructions, commands, information, signals, bits, symbols, and chips referenced herein can be represented by voltages, currents, electromagnetic waves, magnetic fields or particles, optical fields or particles, other items, or a combination of the foregoing.

While the disclosure has been described in connection with certain embodiments and implementations, it is to be understood that the disclosed technology is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

As used in this disclosure, an initial element described by a word or phrase, followed by the phrase “includes at least one of” and one or more additional elements described by one or more words or phrases (which can also include the term “and”) can be construed to mean that the initial element includes any combination of the one or more additional elements. For example, the statement “X includes at least one of A and B” can mean: the initial element X can include the additional element A; the initial element X can include the additional element B; or that the initial element X can include both of the additional element A and the additional element B.

What is claimed is:

1. A method for estimating heart rate with a wearable device, comprising:
receiving movement data and heart rate data, wherein the movement data is indicative of physical exertion of an individual associated with the wearable device, and the heart rate data is measured for the individual during the same period;
determining an estimated human power output based on the movement data;
determining a heart rate demand value for improving a heart rate estimate based on the estimated human power output and at least one adaptive parameter, wherein the heart rate estimate corresponds to the heart rate data, and the at least one adaptive parameter is adjustable based on the heart rate demand value and the heart rate estimate; and
determining an improved heart rate estimate for the individual based on the heart rate demand value and the heart rate estimate.

2. The method of claim 1, wherein the movement data is associated with at least one of: acceleration, velocity, position, and altitude.

3. The method of claim 1, wherein receiving movement data and heart rate data comprises:
receiving, from a first sensor, movement data indicative of physical exertion of the individual associated with the wearable device, wherein the first sensor is at least one of an accelerometer, a barometric pressure sensor, and a Global Positioning System (GPS) sensor; and
receiving, from a second sensor, heart rate data measured for the individual during the same period, wherein the second sensor is at least one of an electrocardiography (ECG) sensor, a photoplethysmogram (PPG) sensor, a pulse oximeter, and an infrared (IR) sensor.

4. The method of claim 1, further comprising:
determining the improved heart rate estimate based on a predicted heart rate change and an improved heart rate estimate determined for a previous period, wherein the predicted heart rate change is determined based on the heart rate demand value and the at least one adaptive parameter.

5. The method of claim 1, wherein the at least one adaptive parameter comprises a scaling parameter and at least another parameter associated with a physiological profile learned from the individual.

6. The method of claim 5, wherein the physiological profile learned from the individual is associated with physical activity models corresponding to activities performed by the individual.

7. The method of claim 1, wherein determining an estimated human power output based on the movement data of the individual comprises:
determining an activity currently performed by the individual based on the movement data; and
selecting, based on the activity, a physical activity model for determining the estimated human power output.

8. The method of claim 1, wherein determining an estimated human power output based on the movement data comprises:
determining the estimated human power output based on a velocity, a gradient value and a mass value, wherein the mass value is associated with the individual, the gradient value is indicative of an inclination of terrain, and at least one of the velocity and the gradient value is derived from the movement data.

9. The method of claim 1, wherein determining a heart rate demand value for improving a heart rate estimate based on the estimated human power output and at least one adaptive parameter comprises:
determining the heart rate demand value based on the estimated human power output, a maximum heart rate, a rest heart rate, and a fitness level.

10. The method of claim 9, wherein the at least one adaptive parameter comprises a scaling parameter related to human power output produced at maximum oxygen effect.

11. The method of claim 10, wherein the fitness level is determined based on the estimated human power output and the scaling parameter, and the scaling parameter is adjusted for the individual over time.

12. The method of claim 1, wherein determining an improved heart rate estimate for the individual based on the heart rate demand value and the heart rate estimate comprises:
determining the improved heart rate estimate using the heart rate estimate and a heart rate estimate based on the heart rate demand value; and
adjusting the at least one adaptive parameter based on comparing the heart rate estimate and the heart rate estimate using adaptive learning.

13. A wearable device, comprising:
a body configured to be coupled to a portion of an individual;
a non-transitory memory; and
a processor configured to execute instructions stored in the non-transitory memory to:
receive movement data and heart rate data, wherein the movement data is indicative of physical exertion of an individual associated with the wearable device, and the heart rate data is measured for the individual during the same period;
determine an estimated human power output based on the movement data;
determine a heart rate demand value for improving a heart rate estimate based on the estimated human power output and at least one adaptive parameter, wherein the heart rate estimate corresponds to the heart rate data, and the at least one adaptive parameter is adjustable based on the heart rate demand value and the heart rate estimate; and
determine an improved heart rate estimate for the individual based on the heart rate demand value and the heart rate estimate.

14. The wearable device of claim 13, wherein the instructions to receive movement data and heart rate data comprise instructions to:
receive, from a first sensor, movement data indicative of physical exertion of the individual associated with the wearable device, wherein the first sensor is at least one of an accelerometer, a barometric pressure sensor, and a Global Positioning System (GPS) sensor; and
receive, from a second sensor, heart rate data measured for the individual during the same period, wherein the second sensor is at least one of an electrocardiography (ECG) sensor, a photoplethysmogram (PPG) sensor, a pulse oximeter, and an infrared (IR) sensor.

15. The wearable device of claim 13, wherein the processor is further configured to execute instructions stored in the non-transitory memory to:
determine the improved heart rate estimate based on a predicted heart rate change and an improved heart rate estimate determined for a previous period, wherein the predicted heart rate change is determined based on the heart rate demand value and the at least one adaptive parameter.

16. The wearable device of claim 13, wherein the instructions to determine an estimated human power output based on the movement data comprise instructions to:
determine an activity currently performed by the individual based on the movement data; and
select, based on the activity, a physical activity model for determining the estimated human power output.

17. The wearable device of claim 13, wherein instructions to determine an improved heart rate estimate for the individual based on the heart rate demand value and the heart rate estimate comprise instructions to:
determine the improved heart rate estimate using the heart rate estimate and a heart rate estimate based on the heart rate demand value; and
adjust the at least one adaptive parameter based on comparing the heart rate estimate and the heart rate estimate using adaptive learning.

18. A system, comprising:
a measurement component, comprising:
a body configured to be coupled to a portion of an individual;
a motion sensor coupled to the body, configured to measure movement data; and
a heart rate sensor coupled to the body, configured to measure heart rate data; and
an analysis component, comprising:
a non-transitory memory;
a processor configured to execute instructions stored in the non-transitory memory to:
receive the movement data and the heart rate data measured for the individual during the same period;
determine an estimated human power output based on the movement data;
determine a heart rate demand value for improving a heart rate estimate based on the estimated human power output and at least one adaptive parameter, wherein the heart rate estimate corresponds to the heart rate data, and the at least one adaptive parameter is adjustable based on the heart rate demand value and the heart rate estimate; and
determine an improved heart rate estimate for the individual based on the heart rate demand value and the heart rate estimate.

19. The system of claim 18, wherein the motion sensor is at least one of an accelerometer, a barometric pressure sensor, and a Global Positioning System (GPS) sensor, and the heart rate sensor is at least one of an electrocardiography (ECG) sensor, a photoplethysmogram (PPG) sensor, a pulse oximeter and an infrared (IR) sensor.

20. The system of claim 18, wherein the processor is further configured to execute instructions stored in the non-transitory memory to:
determine the improved heart rate estimate based on a predicted heart rate change and an improved heart rate estimate determined for a previous period, wherein the predicted heart rate change is determined based on the heart rate demand value and the at least one adaptive parameter.
